(19) United States
(12) Patent Application Publication
(10) Pub. No.: US 2006/0065273 A1
(43) Pub. Date: Mar. 30, 2006

(54) X-ray marker for medical drapes

(75) Inventor: Andrea Lewis, Alpharetta, GA (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/950,739

(22) Filed: Sep. 27, 2004

(51) Int. Cl. A61B 19/00; A61F 5/37

(52) U.S. Cl. 128/849

(57) ABSTRACT

A drape, system, and method of using the system of identifying the location and/or size of an abnormality within a patient, the drape, system, and method each utilizing a x-ray detectable graduated element or marker. Utilization of the present invention is adapted to enhance the ability to detect the size and/or location of an abnormality in a patient thereby allowing a clinician to make better informed decisions concerning the care of the patient.

Each year thousands of patients requiring surgery will have X-rays taken as part of the diagnosis, and/or pre-operative procedures. Some will even have x-rays taken during a procedure. While x-rays can provide a great deal of useful information to clinicians, the location and/or size of an abnormality or defect detected in the x-rays is often difficult to quantify without the benefit of additional testing and/or an invasive procedure. Alternatively, clinicians had to use rulers or the like held up to an x-ray in an attempt to measure or determine the position of the abnormality.

Although many clinicians have the benefit of obtaining an x-ray prior to a procedure, x-ray films are not always readily available or practical for real time feedback. For example, during some procedures such as an angiogram, a clinician will frequently use an x-ray device to generate an image or picture on a display screen. However, due to the need for continuous or real time feedback, the production of x-ray films may not be practical in many such environments. Furthermore, it is common for the display screens to provide an enlarged image which is not of actual size because of the size of the body parts involved. Thus, the ability to estimate the location or size of an abnormality within a patient requires the clinician to know the ratio of enlargement on the screen as the proportions of particular portion of anatomy can vary greatly between two different patients.

In years past, many surgical techniques required much larger surgical incisions than that generally necessary for today's procedures. Accordingly, even if the clinician knew exactly where an abnormality in the patient existed, the incision frequently had to be significantly larger than that needed to merely access the area of the abnormality or defect. With the advances in medical technology and medical procedure, the size of the incision is not always a controlling factor as to the location of the incision.

Thus, if a clinician were able to know with at least some particularity the size and/or location of the abnormality requiring the invasive procedure, the clinician might be able to use and/or make a smaller, more precision incision. Smaller incisions generally offer the benefits of less trauma, faster healing, smaller opportunity for infection, less surgery time, and faster recovery.

In current practice, several surgical products, such as swabs and sponges, include x-ray detectable markers. However, the markers found on those surgical products are used to locate missing sponges or other surgical products at the end of a procedure. Standard operating room procedures require all materials brought into the operating room to be accounted for upon the completion of the procedure. It is thus common practice in the medical field to include a radiopaque marker on all surgical sponges so that the presence or absence of a sponge in a patient may be determined by conducting an x-ray and review of the x-ray films rather than re-opening or reoperating on the patient in those instances when a sponge or the like is initially unaccounted for and which may have been inadvertently left in the patient. Exemplary x-ray detectable markers in surgical sponges are disclosed in EP 0 272 901 B2, DE-A-2 600 185, and GB-A-Patent 858787. Again, this type of x-ray detectable marking is present only to assist in determination as to the existence of the product within the patient and not to assist in the determination as to the location or the size of an abnormality in a patient.

While rulers to measure distance have been used in the operating room, their use in an x-ray either on a film or a real time display on a monitor heretofore has been not beneficial and in fact limiting as the rulers have been made of materials which do not show up in an x-ray produced image and can offer no benefit as they are not visible therein.

None of the prior products or devices provide a scaled or graduated marking which will appear in an x-ray display (film or video screen) and which assists a clinician with the determination of the position and size or length of blockage or abnormality. As such a determination could assist the clinician in determining the type of procedure to be performed, the type or size of instrument to be used, the extent of treatment, the location of additional incisions, as well as provide for a guide for a precision surgery, and/or the like, there is a need and desire for such a product to provide real value guidance or measurements to a clinician.

As such, there is a need and desire for a product or system which may be used during x-ray diagnostics which will facilitate a clinician's determination as to the location, size, length and/or the like of an abnormality (e.g., arterial blockage, tumor, etc.) within the patient, thereby assisting the clinician in determining the type of procedure to be performed, the type or size of instrument to be used, the extent of treatment, the location of additional incisions, as well as provide for a guide for a precision surgery, and/or the like.

### SUMMARY OF THE INVENTION

In response to the difficulties and problems discussed above, a surgical drape and system including a drape has been developed which provides for the use of graduated x-ray detectable markings therewith.

One aspect of the present invention is directed to a medical drape with an x-ray detectable graduation. The drape includes a substrate, and a plurality of x-ray detectable elements, wherein the x-ray detectable elements are graduated. The graduated elements are adapted to facilitate identification of the position and/or size of an abnormality seen during x-ray diagnostics.

The present invention is also directed to a system of accurately identifying the location and/or size of an abnormality during x-ray diagnostics. The system includes a surgical drape, and at least one radiopaque marker, the marker having or being positioned so as to form a regular graduation along a portion of the drape, wherein the at least one radiopaque detectable marker is adapted to enhance the ability to detect under radiological exposure the size and/or location of an abnormality in a patient.

The present invention is also directed to a method of providing a system of identifying the location and/or size of an abnormality during x-ray diagnostics, including the steps of: providing a system including a drape and at least one radiopaque marker, and providing directions for positioning the drape and at least one radiopaque marker relative to the patient prior to an x-ray diagnostic procedure so as to allow the marker to be visibly revealed in an x-ray produced image, thereby enabling a clinician to accurately determine the location and/or size of abnormality revealed in the image.

The method of the present invention may further include the step of utilizing the x-ray produced image during a medical procedure to select or identify the proper point of treatment, an appropriately sized instrument for the procedure, the size of incision, and/or the extent of treatment.

The invention will be more fully understood and further features and advantages will become apparent when reference is made to the following detailed description of exemplary aspects of the invention and the accompanying drawings.

### BRIEF DESCRIPTION OF THE DRAWINGS

The purpose and advantages of the present invention will be apparent to those skilled in the art from the following detailed description in conjunction with the appended drawings in which:

FIG. 1 is a top view of an aspect of a surgical drape according to the present invention, the drape being shown positioned across a representation of a portion of human torso.

### DETAILED DESCRIPTION OF THE PRESENT INVENTION

Reference will now be made in detail to one or more examples of the invention depicted in the figures. Various elements of the present invention will be given numeral designations and the invention will be discussed so as to enable one skilled in the art to make and use the invention. It should be appreciated that each example is provided by way of explaining the invention, and not as a limitation of the invention. For example, features illustrated or described with respect to one aspect may be used with another aspect to yield still a further aspect. These and other modifications and variations are contemplated to be within the scope and spirit of the invention.

In addition, the invention will be described in the context of its various configurations. It should be appreciated that alternative arrangements of the invention can comprise any combination of such configurations. As such, the use of a desired aspect for ease in understanding and describing the invention shall not limit the scope of the invention in any manner.

For ease in understanding, the following detailed description will be made in the context of a surgical drape which is adapted for use in a medical and, more specifically, a surgical environment. It should be appreciated that, although the present invention has particular usefulness with surgical drapes in general, the invention is not limited in scope to surgical drapes but also extends to medical drapes in general.

As used herein, the term x-ray is intended to include not only x-ray or x-ray procedures, but also other high energy techniques which may be used for radiographic visualization. Generally, x-rays utilize wavelengths of less than about 100 angstroms, however, as above, the present invention also contemplates other suitable wavelengths, including those which fall in gamma, infrared and the like classifications.

Further still, while the substrate of some drapes may be minimally radiopaque so as to cause a haze to appear in an x-ray image, the term radiopaque as used herein in connection with the graduated elements or markers is intended to refer to materials with radiopacity greater than that of a drape they are included in or securable to.

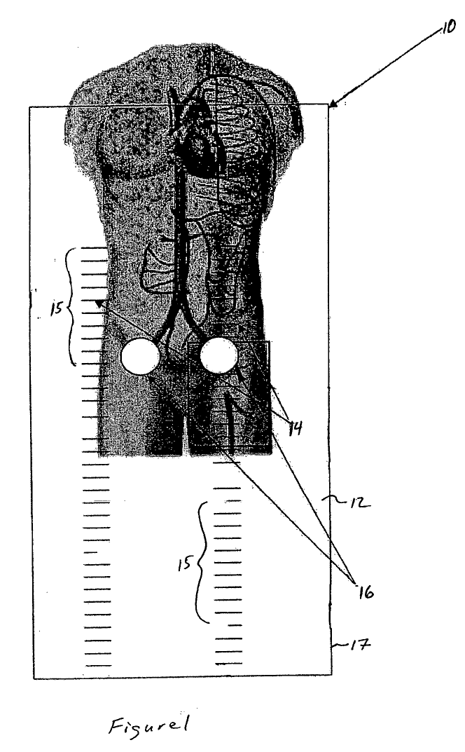

FIG. 1 illustrates a drape 10 according to the invention. Such a drape has wide application and, while the invention may be useful in surgical applications, the invention can be used in any instance where it is desirable to more precisely identify the size and/or location of an abnormality or defect (e.g., growth, clot, etc.) within a patient as depicted on or in an x-ray image. The drape 10 illustrated includes a substrate 12 and a plurality of x-ray detectable (i.e., radiopaque) elements 14. The substrate can be made of any number of suitable materials including for example, woven or nonwoven materials. Exemplary suitable nonwoven materials include, spunbond, meltblown, films, foams, or the like. It will be appreciated that the substrate can be made of one or more of the nonwoven materials or a combination thereof. Still further the drape can consist of different panels or sections each having a composition which is the same or different than the panels or sections adjacent thereto. The different panels or sections may be selected for inclusion in a drape or portion thereof depending on the material characteristics (e.g., permeability, absorbency, liquid repellency, etc.) thereof. Although optional, a surgical drape may include one or more fenestrations or openings 16, as shown in FIG. 1.

The graduated x-ray detectable elements 14 may comprise any number of suitable materials which are visible in an x-ray produced image, whether the image is produced on a film or in real time on a display monitor or video screen. It will be appreciated that the x-ray detectable elements 14 may form a component such as a marker 15 which is graduated by the elements 14. Exemplary materials which may be used to create the elements 14 and/or marker 15 include barium, iodine, gadolinium, or the like. The marker 15 may be one piece or component (e.g., if printed or if cut as one piece), or it may be a plurality of components (e.g., threads, printed lines, or the like) which are positioned at regularly spaced intervals so as to create a graduated marker 15 as illustrated in FIG. 1. The graduated x-ray detectable element(s) 14 which form the marker may be secured to, attached to, or otherwise capable of communication (fixed or detachable) with the substrate 12 or a portion thereof in any suitable manner.

Exemplary ways of placing the x-ray detectable elements 14 in communication with the substrate 12 of the drape 10 so as to form a graduation or graduated marker 15 include, but are not limited to, lamination, gluing, taping, printing, spinning, or otherwise adhering. Depending on the manner in which the graduated elements 14 are formed on or in the drape 10, the elements 14 or marker 15 may be fixed or relocatable. In those aspects of the present invention in which the graduated elements 14 or a marker in which they are present is releasably securable to the drape, the clinician has the ability to place the graduated scale in the most desired orientation or direction relative to the patient and the abnormality being addressed. The ability to relocate the elements 14 or a marker may provide for more accurate size and/or location determinations than if the elements 14 or marker 15 were positioned off-axis from the object or abnormality of interest.

The present invention contemplates the inclusion of series of x-ray detectable elements or a plurality of radiopaque markers having a variety of scales of graduation (e.g., inches, centimeters, millimeters, etc.). It should be appreciated that the size of the graduated scales may be selected based on the size of the drape and/or the type of procedure contemplated. It will be appreciated that a plurality of graduated series of x-ray detectable elements or multiple graduated markers may be included in a drape and/or that the graduation of the markers or of the series of x-ray detectable elements may vary within a drape.

It is contemplated that the dimensions may be of any suitable size or dimension, that the length of the graduated markings may cover the entire length or Width of a drape or a just a portion thereof, and that the graduated markings may extend across multiple sections or panels of a drape. Nevertheless, the images produced by the elements 14 or marker are desirably of such dimensions that, while visible to a clinician in an x-ray, that they do not impede or significantly impede the view of the contrast of the x-ray used to highlight bones or defects/abnormalities in arteries or veins during the x-ray. That is, where at least a portion of the marker 15 or elements 14 are to overlay an area of interest within the x-ray, a marker or element should be selected having suitably small dimensions or configurations so as not to unduly or significantly interfere with the contrast of the x-ray of the area of interest. This selection may be done by the manufacturer based on the expected use of a particular drape. Of course, the graduated markings may be present along the edge of the drape or an edge of a portion of the drape which is adjacent the area of interest within the x-ray field. It will be appreciated that a detachable marker of the type discussed above may desirable in this instance.

The materials used to form the graduated markings should be selected so as not to blend with a contrast fluid which may be used in certain procedures for visualization of blockages, etc. in a patient.

It is further contemplated the drapes of the present invention may be made one or more panels or sections and that the drapes may include one more fenestrations 16. In most aspects of the present invention, the panels or sections of the drapes will include, at least in part, a nonwoven material such as spunbond fibers, meltblown fibers, films, foams, or a combination thereof such as spunbond/meltblown (SM), spunbond/meltblown/spunbond (SMS), film, film/meltblown, film/spunbond, foam/film, film/SM, film/SMS, etc.

The present invention is also directed to a system of accurately identifying the location and/or size of an abnormality during a procedure involving x-ray diagnostics. The system generally includes a surgical drape 10, and at least one radiopaque marker 15, with the marker 15 having or being positioned so as to form a regular graduation along a portion of the drape 10. As above, the graduation may be anywhere on the drape 10, including for example along an edge 17 of the drape 10 or in the middle of the drape 10. Each radiopaque detectable marker 15 is adapted to enhance the ability to detect under radiological exposure the size and/or location of an abnormality in a patient. Again, such an ability is especially useful where x-ray films are not taken or readily available, but rather where real time display of the results of the radiographic exposure is displayed on a video monitor, and more specifically, in those instances where the display is not to-scale.

As with the x-ray detectable elements discussed above, the marker(s) of the present invention may be incorporated into or on the drape. It is contemplated that each radiopaque marker may be laminated, adhered, taped, printed, spun or otherwise formed or secured on or in the drape.

As above, any suitable radiopaque material is contemplated. Exemplary materials for use in the at least one radiopaque marker include barium, iodine, gadolinium, and the like.

The system of the present invention also contemplates that the graduated radiopaque marker 15 may be removably securable to the drape 10. A removably securable graduated radiopaque marker 15 allows a clinician to reposition the marker about the drape 10 so that the marker 15 is in the desired location and is “on-axis” relative to the growth or abnormality, thereby allowing more a more accurate determination of the size and/or location of the abnormality.

The present invention is also directed to a method of providing a system of identifying the location and/or size of an abnormality during x-ray diagnostics. The method generally includes the steps of providing a system similar to that discussed above (e.g., having a drape and at least one radiopaque marker), and providing directions for positioning the drape and the radiopaque marker(s) relative to a patient prior to an x-ray diagnostic procedure such that the marker may be visibly revealed in the x-ray produced image. The revealed image then may enable a clinician to accurately determine the location and/or size of abnormality revealed in the image. At least one aspect of a method of the present invention also contemplates that after the clinician has determined the location and/or size of abnormality revealed in the image using the in part the radiopaque graduations of the drape that the clinician may use the x-ray image produced during the medical procedure to select or identify: the proper point of treatment, an appropriately sized instrument for the procedure, the size of incision, and/or the extent of treatment.

An exemplary use of the system and the method of the present invention is during a femoral angiogram procedure. In such a procedure, a drape having a fenestration for entry into the femoral artery of the patient is appropriately placed over the patient. A catheter having a radiopaque marking near the tip is inserted into the patient's femoral artery and passed therein towards the location of a suspected clot or occlusion. During such a procedure it is common to have a live or real time video display from an x-ray machine, where the display shows a portion of the patient where the suspected occlusion exists, as well as the radiopaque catheter tip. As the video display is usually enlarged, the actual distance between the tip of the catheter and the blockage and/or the size of the blockage may be difficult for the clinician to determine. The present invention offers the advantage of the graduated radiopaque marker and allows the clinician to determine how much farther the catheter needs to be advanced so the procedure can be performed, allows the clinician to determine when the procedure may be stopped, whether another procedure may need to be performed, and/or whether another instrument needs to be used, and reduces the likelihood a clinician will incorrectly estimate the distance on a enlarged display in real time where no graduation is present. The ability of a clinician to make a more informed decision allows for a reduction in potential damage to or further risk to the patient as compared to prior procedures that do not provide such location or size information.

From the above discussion, it will be appreciated that a radiopaque marker may be included either in the drape or may be attached thereto such that when an x-ray is taken during the procedure it is in the field of the display. If it is not, and the graduated marker is detachable the marker may merely be repositioned and reattached in position which provides the desired view in the display.

While the invention has been described in detail with respect to specific aspects thereof, those skilled in the art, upon obtaining an understanding of the invention, may readily conceive of alterations to, variations of, and equivalents to the described aspects and the processes for making them. The invention may be embodied in other specific forms without departing from the scope and spirit of the inventive characteristics thereof. The present aspects therefore are to be considered in all respects as illustrative and not restrictive, the scope of the invention being indicated by the appended claims rather than by the foregoing description, and all changes which come within the meaning and range of equivalency of the claims are therefore intended to be embraced therein.

I claim:

1. A medical drape with an x-ray detectable graduation, the drape comprising:
a substrate; and
a plurality of x-ray detectable elements located on said substrate;
wherein the x-ray detectable elements are graduated, the graduated elements being adapted to facilitate identification of the position and/or size of an abnormality seen during x-ray diagnostics.

2. The drape of claim 1, wherein the substrate is at least in part a nonwoven substrate.

3. The drape of claim 1, wherein the substrate is comprised at least in part of a nonwoven material, a film, a foam, or a combination thereof.

4. The drape of claim 1, wherein the x-ray detectable elements are selected from the group comprising barium, iodine, gadolinium, or the like.

5. The drape of claim 1, wherein the x-ray detectable elements are laminated, adhered, taped, printed, spun or otherwise formed or secured on or in the substrate.

6. The drape of claim 1, wherein the drape further comprises at least one fenestration.

7. The drape of claim 1, wherein the drape comprises a plurality of graduated series of x-ray detectable elements.

8. The drape of claim 7, wherein the graduated series of x-ray detectable elements have different scales of graduation.

9. The drape of claim 1, wherein the graduated x-ray detectable elements are detachable from the substrate.

10. The drape of claim 9, wherein the detachable x-ray detectable elements are repositionable on the drape.

11. A system of accurately identifying the location and/or size of an abnormality during x-ray diagnostics, the system comprising;
a surgical drape; and
at least one radiopaque marker, the marker having or being positioned so as to form a regular graduation along a portion of the drape;
wherein the at least one radiopaque detectable marker is adapted to enhance the ability to detect under radiological exposure the size and/or location of an abnormality in a patient.

12. The system of claim 11, wherein the at least one marker is incorporated into or on the drape.

13. The system of claim 11, wherein the surgical drape is comprised at least in part of a nonwoven material, a film, a foam, or a combination thereof.

14. The system of claim 11, wherein the surgical drape has multiple panels.

15. The system of claim 11, wherein the at least one radiopaque marker is selected from the group comprising barium, iodine, gadolinium, or the like.

16. The system of claim 11, wherein the at least one radiopaque marker is laminated, adhered, taped, printed, spun or otherwise formed or secured on or in the substrate of the material.

17. The system of claim 11, wherein the drape further comprises a fenestration.

18. The system of claim 11, wherein the graduated radiopaque marker is a removably securable to the drape.

19. The system of claim 11, wherein the graduated radiopaque marker is repositionable about the drape.

20. A method of providing a system of identifying the location and/or size of an abnormality during x-ray diagnostics, comprising the steps of:
providing a system comprising the system of claim 11, the system including a drape and at least one radiopaque marker; and
providing directions for positioning the drape and at least one radiopaque marker relative to the patient prior to an x-ray diagnostic procedure so as to allow the marker to be visibly revealed in an x-ray produced image;
thereby enabling a clinician to accurately determine the location and/or size of abnormality revealed in the image.

21. The method of claim 20, further comprising utilizing the x-ray produced image during a medical procedure to select or identify:
i) the proper point of treatment;
ii) an appropriately sized instrument for the procedure
iii) the size of incision; or
iv) the extent of treatment.
